(19) United States
(12) Patent Application Publication
(10) Pub. No.: US 2007/0184486 A1
(43) Pub. Date: Aug. 9, 2007

(54) Diagnosis or treatment of endothelial cell dysfunction related diseases

(76) Inventors: Andrius Kazlauskas, Winchester, MA (US); Guilio Romeo, Somerville, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/589,687

(22) PCT Filed: Feb. 22, 2005
(86) PCT No.: PCT/US05/05668

§ 371 Date: Aug. 17, 2006

Related U.S. Application Data
(60) Provisional application No. 60/546,389, filed on Feb. 20, 2004.

(51) Int. Cl. G01N 33/53

(52) U.S. Cl. 435/7.1

(57) ABSTRACT

The present invention provides a method of determining the disease state of a disease or condition associated with EC dysfunction in a subject. An exemplary method screens for elevated profilin-1 levels as a marker for DVD and other vascular diseases such as atherosclerosis, cerebrovascular disease, CVD or peripheral vascular disease. The invention also provides a method for the prophylaxis or treatment of such diseases or conditions associated with EC dysfunction. The method comprises administering to a subject a therapeutic composition or carrying out a protocol to inhibit the activity or function of profilin-1. In an embodiment, the composition comprises an inhibitor of profilin-1 activity or function administered in a pharmaceutically acceptable carrier. The therapeutic protocol or method can, for instance, be siRNA based or a strategy for gene therapy. The invention further provides a kit that can be used to carry out a diagnostic method.

### CROSS REFERENCE TO RELATED APPLICATIONS

This application claims the priority of U.S. Provisional Application No. 60/546,389 filed Feb. 20, 2004 and entitled, DIAGNOSIS OR TREATMENT OF ENDOTHELIAL CELL DYSFUNCTION IN COMPLICATIONS OF DIABETES OR ATHEROSCLEROSIS, which is hereby incorporated by reference herein.

### BACKGROUND OF THE INVENTION

Atherosclerosis and other related vascular diseases such as cardiovascular disease (CVD) are often a complication of diabetes mellitus (DM). Atherosclerosis is the main cause of morbidity and mortality in patients suffering from DM. The acceleration of such vascular diseases in DM patients tends to be multifactorial with an increased incidence of factors that can include obesity, hypertension, hyperglycemia and low levels of high density lipoprotein (HDL) cholesterol playing a role. Although the specific pathogenesis of DM remains unknown, several theories exist as to the cause of the disease. A relatively recent theory is that endothelial cell (EC) dysfunction may likely be involved in the pathogenesis of vascular diseases that are linked to DM including diabetic vascular disease (DVD) and other vascular diseases such as CVD, atherosclerosis, peripheral vascular disease or cerebrovascular disease.

EC dysfunction is generally characterized by, for example, the increased adherence of mononuclear cells to the endothelium, the stimulation of macrophage lipoprotein lipase (LPL) production and overexpression of LPL, accelerated apoptosis, phosphorylation of vasodilator-stimulated phosphoprotein (VASP) and leukocyte adhesion through intercellular adhesion molecules (ICAM-1). By building on the understanding that EC dysfunction can participate in the development of DVD, diagnostic and therapeutic interventions that screen for or target pathogenic events associated with the endothelium could be developed for identifying and treating DVD and vascular diseases like atherosclerosis.

For example, a diagnostic protocol or strategy can be developed by recognizing and characterizing the role EC dysfunction plays in causing DVD and other vascular diseases. Additionally, therapeutic interventions could be used to effectively complement conventional diabetic treatment approaches, which are commonly directed toward controlling blood sugar levels. Such protocols and interventions would also be highly desirable for identifying and targeting early events in the pathogenic cascade that leads toward the late stages of DVD.

### SUMMARY OF THE INVENTION

The present invention provides such diagnostic protocols and therapeutic interventions as identified above. In one embodiment, the invention is directed toward a method of determining the state of a disease or condition associated with EC dysfunction in a subject. The method, for example, screens for levels of profilin-1 as a marker for DVD and other vascular diseases that include peripheral vascular disease, CVD, atherosclerosis or cerebrovascular disease. Such elevated profilin-1 levels can be found in vascular circulations or in aorta and retina endothelium tissues. Profilin-1 is established to be a convenient and accurate marker for studying the progression or onset of DVD and vascular diseases in subjects suffering from or believed to be at risk of suffering from such diseases or conditions like, for example, a patient with a predisposition to DM. The invention is also directed to a kit that can be used to carry out such methods.

Profilin-1 has also been shown to be a practical and effective treatment target for the diseases and conditions associated with EC dysfunction. In another embodiment of the invention, a method is provided for prevention or treatment of these diseases and conditions. The method generally comprises administering to a subject a therapeutic composition or carrying out a protocol to inhibit the activity or function of profilin-1. For example, in one embodiment, the therapeutic composition is an inhibitor. of profilin-1 activity or function administered to a subject in a pharmaceutically acceptable carrier. The therapeutic protocol can be a strategy for gene therapy in which a gene associated with profilin-1 activity or function is modified, substituted, regulated or interfered with. Alternatively, the invention comprises an approach for downregulating transcription of a gene associated with profilin-1 synthesis via the use of small interfering RNA (siRNA) inhibition.

### DESCRIPTION OF THE DRAWINGS

Other features and advantages of the present invention will be apparent from the detailed description of the invention that follows, taken in conjunction with the accompanying drawings of which:

FIG. 1 shows staining of aorta sections from diabetic and nondiabetic rats using phage clone 45;

FIG. 2 shows Western blot analyses for profilin-1 in rat glomeruli and in the aortic endothelium obtained from both a diabetic rat and human; and

FIG. 3 shows staining for profilin-1 in atherosclerotic plaque from apoE-null mice.

### DETAILED DESCRIPTION OF THE INVENTION

Unless otherwise stated, the following definitions provide meaning and examples to terms used herein. These definitions are also intended to encompass any meaning that would be contemplated by a person of ordinary skill within the art.

The terms “elevated” or “increased” with regard to levels of profilin-1 in a subject generally refer to profilin-1 levels that exceed or are above those that would normally be expected in a subject that does not have a predisposition for or does not suffer from a condition or disorder associated with EC dysfunction like, for example, DVD, atherosclerosis or CVD.

The terms “prophylaxis” or “prevention” generally refer to measures intended to preserve the health of a subject and are not intended to relate to a cure of a condition or disorder associated with EC dysfunction.

The terms “diagnosis” or “diagnosing” generally refer to identifying or determining whether a subject suffers from or is likely to suffer from a disease or condition associated with endothelial cell dysfunction such as, for example, peripheral vascular disease, CVD, atherosclerosis, cerebrovascular disease or DVD.

The term “positive diagnosis” generally refers to a determination that a subject suffers from or is likely to suffer from a disease or condition associated with endothelial cell dysfunction such as, for example, DVD, peripheral vascular disease, atherosclerosis, CVD or cerebrovascular disease.

The investigation leading to the present invention involved identifying surface targets or markers that are differentially expressed on the endothelial tissues of a diabetic subject. The investigation was also directed toward evaluating and controlling the activity or function of such surface targets in the diseases or conditions associated with EC dysfunction. These diseases and conditions include, for example, peripheral vascular disease, DVD, atherosclerosis, cerebrovascular disease or CVD. In the experimental procedures and examples that are described below, it was found that profilin-1 is an effector of EC dysfunction. Profilin-1 was also found to be a convenient and accurate marker and treatment target for diseases and conditions associated with EC dysfunction. Based on these findings, the invention provides methods for both the diagnosis and treatment of such diseases and conditions.

In particular, profilin-1 was found to interact in vitro with a binding partner identified to be phage clone 45. A direct consequence of such interaction is that profilin-1 can translocate in vivo onto a cell surface when overexpressed. Phage clone 45 was also found to be aorta and diabetic specific in a subject. Such specificity resulted in profilin-1 levels being significantly increased above normal in the aorta endothelium of diabetic rats. Given that profilin-1 levels are shown to be increased in aorta tissues of diabetic subjects and, as described in detail below, in atherosclerotic plaque, cell surface localization of profilin-1 can be used as a marker for injured EC or as a therapeutic target for preventing the onset or progression of diseases and conditions that are associated with EC dysfunction. It is also recognized that the diabetic rat model used for these experimental procedures and examples is a useful and accurate predictor of human DVD.

As further supported by the identified role of profilin-1 in EC dysfunction as associated with DVD, it was shown that neither high glucose concentrations, cytokine tumor necrosis factor-alpha (TNF-α) nor recombinant profilin-1 upregulated profilin-1 levels. Conversely, it was shown that low density lipoprotein (LDL) and, in particular, oxidized LDL could increase profilin-1 at both the mRNA and protein levels. Similarly, oxidized cholesterol or oxysterol was also shown to be able to upregulate profilin-1 in rat aorta EC (RAEC). With LDL and oxysterol being primary risk factors for CVD and other vascular diseases, profilin-1 likely operates downstream of such factors in mediating CVD.

The examples that follow show the physiological consequences of increased levels of profilin-1 in, for example, a diabetic aorta. Such elevated or increased profilin-1 levels have been shown to accelerate apoptosis, phosphorylate VASP and promote leukocyte adhesion through, for example, ICAM-1. These findings indicate that profilin-1 mediates endothelial cell dysfunction through several mechanisms. moreover, the findings demonstrate that profilin-1 is a marker and treatment target for such diseases or conditions as DVD, peripheral vascular disease, atherosclerosis, CVD, cerebrovascular disease. It is appreciated that mechanisms including an increased adherence of mononuclear cells to the endothelium, stimulation of macrophage LPL production and ovrerexpression of LPL can also be linked to elevated levels of profilin-1.

Profilin-1 expression is further shown to be increased in atherosclerotic plaque. This finding used a validated mouse model for human atherosclerosis that displayed elevated levels of LDL and very low density lipoprotein (VLDL). The elevated levels of LDL and VLDL were also shown to be enhanced by DM. The model also indicated that profilin-1 expression was dramatically upregulated in atherosclerotic lesions when compared to adjacent areas that were lesion-free. In addition, labeling also revealed that profilin-1 was overexpressed in EC within the plaque and macrophage infiltrating the plaque.

The evidence presented herein indicates that EC dysfunction underlies and precedes the structural alterations of DVD and other vascular diseases (1, 2 and 3). EC dysfunction has further been identified with a defect in both endothelial dependent and NO-mediated vasorelaxation. Defects of this type generally precede an overt diabetic state (1 and 3). LDL abnormalities in a diabetic subject further tend to include an increase in highly atherogenic dense and minimally oxidized LDL, which contributes to heightened susceptibility for DM complications such as atherosclerosis (4 and 5). LDL levels similarly correlate with endothelial function even within normal ranges, indicating that lipid-mediated injury is an early event for the onset and progression of CVD (6).

In one embodiment, the invention is directed toward a method of determining the state of a disease or condition associated with EC dysfunction. An exemplary method comprises providing a subject suffering from or believed to be at risk of suffering from the disease or condition such as a patient having a predisposition to DM. The method then, for instance, screens at a site in the subject for levels of profilin-1, which are shown to be a positive diagnosis marker when elevated for DVD and other vascular diseases that include, without limitation, atherosclerosis, cerebrovascular disease, CVD or peripheral vascular disease. Such elevated profilin-1 levels can be detected in various sites associated with vascular circulations such as sera or the aorta and retina endothelium.

The invention is also directed to a kit that can be used to carry out a diagnostic method such as the method described above for determining the state of a disease or condition associated with EC dysfunction. An exemplary kit comprises an indicator for indicating a level of profilin-1 in a subject such as an elevated or increased level of profilin-1. The indicator, for example, can be a chemiluminescence-based compound. The kit also comprises a device such as a conventional light detection source for monitoring the indicator qualitatively or quantitatively.

In another embodiment according to the invention, a method for prophylaxis or treatment of a disease or condition associated with endothelial cell dysfunction is provided. An exemplary method of the invention comprises administering siRNA to a subject in order to downregulate gene transcription associated with profilin-1. As described in more detail below, the siRNA can be delivered via a lentivirus. The siRNA is, alternatively, delivered to, for example, a coronary, carotid or femoral artery to prevent or treat the disease or condition. The siRNA can also be a synthetic siRNA.

The invention further contemplates using in vivo methods for studying or diagnosing profilin-1 expression on the surface of EC in the aorta, femoral and carotid arteries of a subject such as a LDL receptor knockout (Ldlr) mouse. Ldlr mice are known to develop atherosclerotic lesions and are understood to be a valid model for human atherosclerotic disease. An exemplary method of the invention takes place over a period of time. In the method, the synthetic peptide cognate to the peptide unique to phage clone 45 is labeled with, for example, a near infrared sensitive dye and injected intravenously. The labeled peptide mimics the binding of phage clone 45 without the untoward effects of injecting a viral particle into vascular circulation. The injection also allows the labeled peptide to bind exclusively to profilin-1 exposed on the surface of endothelium tissues as the peptide cannot enter the intercellular compartment.

Through employing a computer-assisted infrared source, the extracellular profilin-1 in the plaque is detected by monitoring the binding of labeled peptide to the endothelium of the aorta, femoral or carotid artery of the subject. The method according to the invention is used to assess the spatial relationship between extracellular profilin-1 in the endothelium and the development of atherosclerotic lesions. A longitudinal method of assessment also addresses whether extracellular profilin-1 correlates with plaque progression in a subject over a certain period of time.

Also able to be studied by a method of the invention are circulating levels of profilin-1 and antibodies anti-profilin, which are specific markers for human DVD. For example, a subject like one of the rats or mice that are described below is used to investigate circulating levels of profilin-1 and also to diagnosis whether the subject suffers from or is likely to suffer from a disease or condition associated with EC dysfunction. The method is also used to assess circulating levels of profilin-1 in normal subjects affected by DM regardless of whether the subject has a background of DM or other vascular diseases.

In particular, sera can be acquired from the categories of diabetic subjects that include patients without a disease or condition associated with EC dysfunction, patients with a background of angina, transitory ischemic attack or claudicatio intermittens, patients with prior significant cardiovascular events including myocardial infarctions, strokes or limb amputations and patients with various diabetic vascular complications that can include non-proliferative retinopathy or kidney disease. Circulating profilin-1 levels can similarly be assessed by a method according to the invention using enzyme-linked immunosorbent assays (ELISA). Data collected through such methods can establish whether circulating profilin-1 is increased in a diabetic subject, such increases correlate with the severity of vascular complications or whether that profilin-1 is a marker for the progression of cerebrovascular disease, peripheral vascular disease, CVD, atherosclerosis or DVD.

In another embodiment, the invention is directed toward a method for prophylaxis or treatment of a disease or condition associated with EC dysfunction. The method comprises providing a subject suffering from or thought to be at risk of suffering from a disease or condition associated with endothelial cell dysfunction such as a patient with a predisposition to DM. The method also comprises administering to the subject a therapeutic composition or carrying out a protocol to inhibit the activity or function of profilin-1.

For example, in one embodiment, the composition can be an inhibitor of profilin-1 activity or function administered to the subject in a pharmaceutically acceptable carrier. The compositions can be administered orally, topically or parenterally, such as intranasally, subcutaneously, intramuscularly, intravenously or intra-arterially, by conventional methods in pharmaceutically acceptable carrier substances. The compositions of the invention could, for instance, be administered in a sustained release formulation using a biodegradable biocompatible polymer or by on-site delivery using micelles, gels or liposomes. The therapeutic compositions can be administered, for example, in a dosage amount from about 0.25 pg per kg per day to about 5 mg per kg per day. Other optimal dosage and modes of administration can further be readily determined by conventional protocols.

In addition, the therapeutic protocol or method can be a strategy for gene therapy in which a gene associated with profilin-1 activity or function is modified, substituted, regulated or otherwise interfered with. Profilin-1 is demonstrated to have a direct and pathogenic role in CVD, peripheral vascular disease, DVD, cerebrovascular disease or atherosclerosis. The nature of such a, role can be used in profilin-1 gene ablation, which can prevent or otherwise treat atherosclerotic lesions in a subject such as Ldlr mice. An exemplary method of the invention can be performed by studying circulating levels of profilin-1, antibodies anti-profilin or through study of profilin-1 extracellular localization in the plaque of an appropriate subject such as Ldlr mice.

The early phases of DVD and other vascular diseases are appropriate for performing a prophylaxis or treatment method of the invention. These early phases of disease onset are often poorly situated for treatment with conventional pharmacological measures. Inhibition therapies or protocols that are focused on profilin-1 being a therapeutic target will be effective for preserving the health of a subject. Exemplary protocols or methods include gene therapy or therapeutic protocols that target the activity or function of profilin-1 or a gene associated with profilin-1 in order to prevent or treat diseases or conditions associated with EC dysfunction.

Alternatively, available models of atherosclerosis such as Ldlr mice are used to develop an in vivo therapeutic method of the invention that is siRNA based. An siRNA based protocol can be employed to blunt or otherwise interfere with the excess of profilin-1 expressed in, for example, atherosclerotic arteries. For instance, such a protocol involves downregulating gene transcription associated with profilin-1 synthesis via siRNA inhibition through using an siRNA oligo corresponding to a specific region of the profilin-1 coding sequence. Such siRNA can also be administered or delivered to a patient to prevent or treat a disease or condition associated with EC dysfunction. The invention also contemplates use of a synthetic siRNA engineered to downregulate gene transcription associated with profilin-1. An siRNA based method of the invention does not completely abrogate profilin-1 gene transcription.

Furthermore, lentivirus can be used to deliver siRNA or synthetic siRNA, without completely abrogating profilin-1 gene transcription. Similarly, the expression of profilin-1 in diabetic rat aorta, where profilin-1 levels are shown to be elevated, can be directly targeted. The therapeutic compositions and protocols of the invention are not intended to reduce or abrogate the constitutive levels of profilin-1 in a subject as profilin-1 is essential for both cell adhesion and proliferation. The compositions or methods of the invention are generally intended to inhibit the activity, function or synthesis of profilin-1 without affecting such essential roles for the protein.

In another embodiment of the invention, a method can be performed concomitantly with the use of a statin therapy for treatment of a vascular disorder. Statins are broadly known to be a family of cholesterol-lowering pharmaceutical agents that tend to prevent atherosclerotic plaque formation in experimental vascular disease models. Statins have also been proven to be beneficial in the prevention and treatment. of human CVD. Thus, by assessing profilin-1 levels in vascular circulation, the efficacy of both established and common anti-atherosclerotic therapies and treatments like statin therapy can be conveniently evaluated. This evaluation would be clinically important as drug efficacy could be monitored over time with minimal expense or invasiveness. In such practice, a method of the invention is a used as a model for the real-time assessment of conventional or experimental treatment or therapy efficacies.

### EXPERIMENTAL PROCEDURES

In order to demonstrate that profilin-1 is an effector of EC dysfunction and, especially, EC dysfunction in humans, four steps in an investigatory protocol were carried out. The steps indicate that profilin-1 is a useful marker and therapeutic target for diseases or conditions that are associated with EC dysfunction. Such diseases or conditions include, for example, atherosclerosis, DVD, cerebrovascular disease, CVD or peripheral vascular disease. The protocol steps are (1) an ex vivo screening of a complex peptide phage display library in diabetic rats, (2) recovery of phage clones including phage clone 45, (3) purification and isolation of phage clone 45 binding partner profilin-1 and (4) validation of binding interaction of phage clone 45 and profilin-1. The rat models used in the protocol described below have been previously validated and are also known to be appropriate for DVD (7 and 8).

### (1) Ex Vivo Screening of A Complex Peptide Phage Display Library In Diabetic Rats

A complex peptide phage display library was screened on isolated EC from a retina and an intact aorta obtained from diabetic rats. Particularly, Sprague-Dawley male rats made diabetic via streptozotocin (55 mg per kg body weight IV) were sacrificed after five months of DM along with age and sex matched control rats. Aortas, retinas and kidneys were isolated from the rats after anesthetizing with ketamine-xylazine (70 mg per kg and 9 mg per kg body weight IM) and perfused with pre-warmed PBS through the left ventricle using a catheter connected to a peristaltic pump for 4 minutes at about 100 mmHg. The thoracic aorta was also harvested and freed of periadventitial fat (9). Furthermore, glomeruli were isolated using a serial sieving technique (10). Fresh retina EC were also isolated by hypotonic shock, a procedure that allows the preferential release of EC from the retinal vascular network and minimizes the loss of cell surface domains because the procedure does not involve tryptic digestion (11). The purity of such preparations were assessed by immunoreactivity to the vonWillebrandt factor (vWF), which is known marker for EC.

The isolated samples were then exposed to a mix of three peptide-phage display libraries with distinct structures of linear SX₈(GS)₄, cyclic SCX₈C(GS)₄ and one structure that included a linear structure followed by a cyclic portion SX₄CX₄C(GS)₄. For each structure, X represents an amino acid, S is invariant to ensure signal sequence cleavage and (GS)₄ is an eight amino acid spacer that is flexible. The complexity of the library was approximately 5×10⁸ with a titer of approximately 1×10¹³ plaque-forming units (PFU) per ml. The peptides were then displayed as fusion to pIII capsid protein of the filamentous phage M13 (12). The library was screened on the intact aorta and isolated retina EC. The initial input of about 3×10¹¹ PFU was added to a screening buffer comprising DMEM phenol-free-hepes (DH) at a pH of about 7.5 and with 0.5% non-fat dry milk. After three washes with the DH buffer, bound phage were eluted with 50 mM glycine at about pH 2.0 for about 5 minutes and neutralized with 100 mM sodium phosphate at about pH 7.4 (9). An aliquot of the elution was then used to titer phage recovery (12). The remainder of such was then amplified in DH5αF′ and used for the next round of selection (13). In total, four rounds of selection were performed.

### (2) Recovery of Phage Clones Including Phage Clone 45

During the fourth round of selection ex vivo, randomly selected clones were tested for their ability to bind EC in retina and aorta tissue sections. In particular, 33 retina and 123 aorta phage clones were individually amplified and used as a reagent to stain respective tissue sections from diabetic and nondiabetic rats (9 and 12). 3×10¹⁰ PFU per section were then used at a 1:20 dilution in DH with 2% non-fat dry milk. The sections were not permeabilized before adding the phage clones. The phage clones were detected with a monoclonal anti-phage M13 antibody followed by an anti-mouse antibody conjugated to Cy3. Stain images were captured and analyzed by conventional computer-assisted hardware and software.

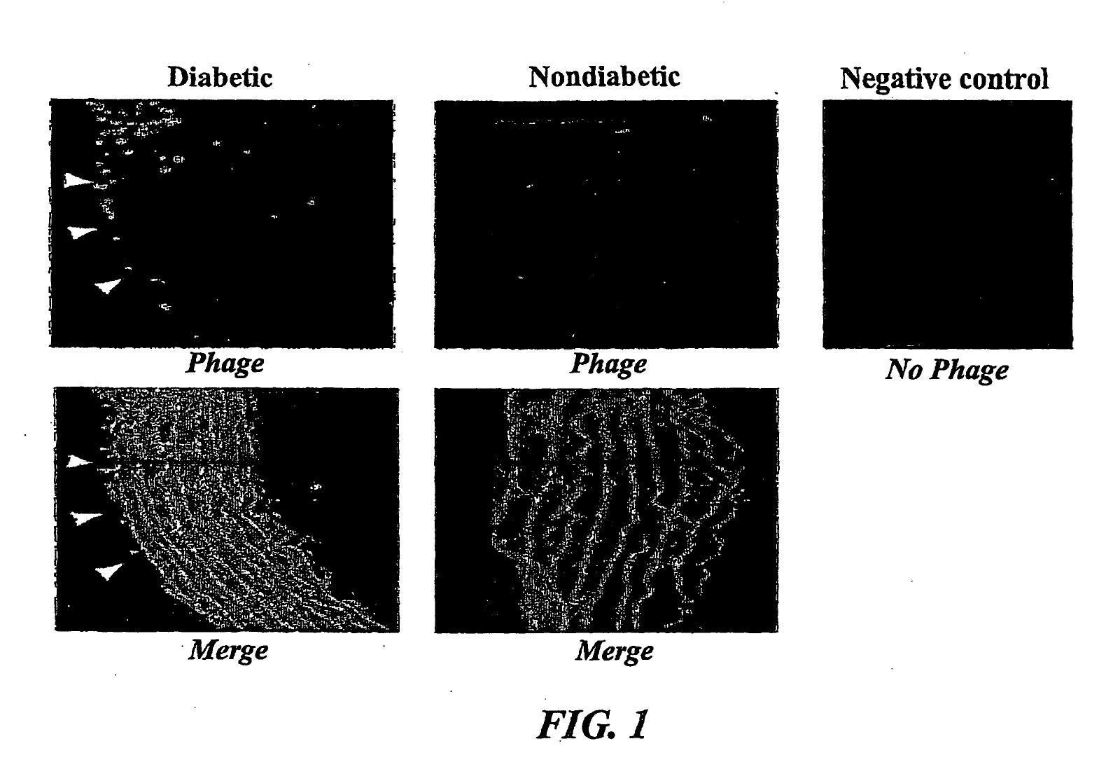

By the selection protocol described above, it was then shown that phage clone 45 isolated from the aorta was able to stain the endothelium of diabetic aorta sections with an intense and uniquely patchy pattern, however, such staining was absent from nondiabetic sections. These results are shown by the stains within FIG. 1. Phage clone 45 did not stain retina or kidney tissue sections. This study shows that phage clone 45 can recognize a conformation on the surface of diabetic aortic EC that is regulated in DM and in an organ specific manner.

The intense and patchy stain pattern of phage clone 45 in FIG. 1 indicates a heterogeneous expression of the binding partner for the clone on the surface of the diabetic endothelium. This finding is in keeping with the established notion that EC exposed to disturbed flow are more likely to develop atherosclerosis and show a discrete program of gene expression (14 and 15). As such, phage clone 45 staining can be used to mark endothelial regions that are susceptible to injury and, accordingly, to atherosclerosis.

### (3) Purification And Isolation of Phage Clone 45 Binding Partner Profilin-1

Via a multi-stage assay provided by the invention, profilin-1 was isolated and purified as a binding partner for phage clone 45 in diabetic aorta tissues. The assay was used to exploit the inherent mass of phage clone 45, which is about 16 MD. Lysates from the aorta endothelium of nondiabetic and diabetic rats were obtained with a sample population (n) equal to 10. 5×10¹² PFU of phage clones including phage clone 45 were then incubated with the endothelial lysates (90′) at about 4° C. on a rotator. A mouse monoclonal anti-phage M13 antibody at about 60 μg was then added to the mixture (20′) at room temperature and spun through a 0.1 μn microporous filter, followed by extensive washing. It had been previously shown that complex antibody-phage could be efficiently retained on a 0.1 μm filter. The retentate or antibody-phage putative binding partner was then diluted with urea at a final concentration of 3 M in order to elute the phage associated proteins, followed by centrifugation over a 100 kD cut-off filter. The 100 kD filter was chosen for phage clone 45 as phage blots indicated that the binding partner for the clone was approximately 15 kD. The sample was subsequently analyzed by 15% SDS-PAGE under denaturing conditions and silver stained using a conventional silver stain gel kit. Relevant bands were thereafter analyzed by microcapillary reverse-phase high-throughput liquid chromatography-mass spectrometry (HPLC-MS) on a Finnigan LCQ DECA quadrupole ion trap mass spectrometer located at the Harvard Microchemistry Facility, Cambridge, Mass.

The silver staining showed a band of about 15 kD in diabetic lysates incubated with phage clone 45 that was not detected in control lysates or diabetic lysates exposed to a retina specific phage clone 23R. The 15kD band indicated that the binding partner was particular to phage clone 45 and diabetic tissue specific. After analysis by microcapillary reverse-phase HPLC-MS, the 15kD band yielded multiple matches for profilin-1, which is commonly known to be an intracellular protein and a major regulator of actin polymerization and endocytosis (16, 17 and 18).

### (4) Validation of Binding Interaction of Phage Clone 45 And Profilin-1

Direct biding interaction between phage clone 45 and profilin-1 was confirmed in vitro via a conventional pull-down assay. The assay showed that phage clone 45, but not phage clone 23R, could associate with recombinant rat profilin-1. To validate profilin-1 as a specific binding partner for phage clone 45, full-length rat profilin-1 subcloned in pQE-30 downstream of 6His-tag was expressed in M15 E. coli. The 6His-tag was graciously provided by Dr. M. Tamura of Kitakyushu, Japan. Profilin-1 was then purified under native conditions by Ni⁺⁺ affinity chromatography. 6His-tagged recombinant profilin-1 at 10 nM and equimolar concentrations of purified glutathione S-transferase (GST) were incubated with phage clone 45 or phage clone 23R at 10¹¹ PFU and subsequently immunoprecipitated with a mouse monoclonal anti-phage antibody (1).

A result of profilin-1 being a binding partner of phage clone 45 is that a pool of the protein translocates on the cell surface when overexpressed or in response to injury. Therefore, profilin-1 is overexpressed in RAEC employing a retroviral-based protocol and the surface localization of the protein can studied by flow cytometry. Such flow cytometry studies indicate that a small pool of profilin-1 can be expressed on an extracellular space and that the pool had an increase of profilin-1 overexpression in RAEC as compared to empty vector-RAEC with n being 3 and a p-value of 0.01. It was also demonstrated via double-staining with propidium iodide and profilin-1 that necrosis was not responsible for the highly positive population of cells (6).

The examples that follow are provided in order to illustrate advantages of the invention that have not been described above and to further assist a person of ordinary skill within the art in performing or using the methods, protocols or compositions of the invention. The examples can also include or incorporate any of the variations or embodiments of the invention that have been described above. Moreover, the embodiments described above may each include or incorporate the variations of any or all other embodiments of the invention. The examples that follow are not intended in any way to otherwise limit the scope of the disclosure herein.

### EXAMPLE I

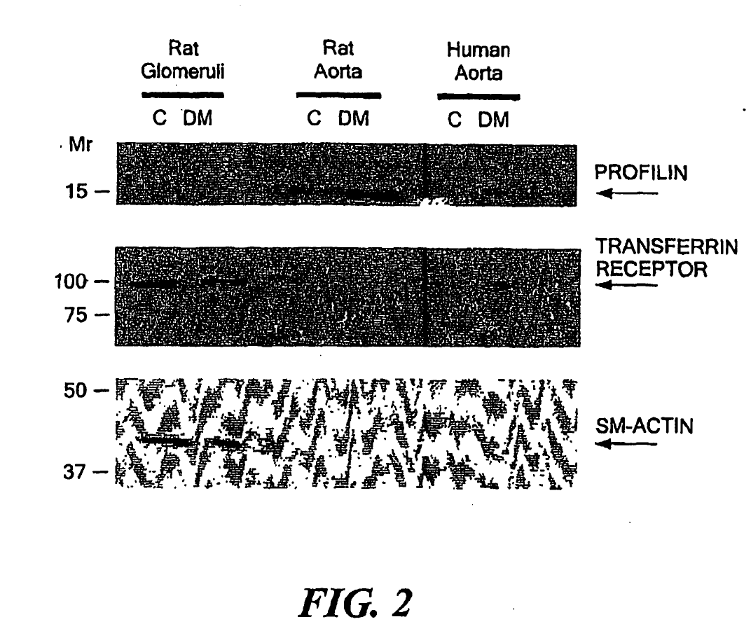

To further determine whether profilin-1 is an effector of EC dysfunction and also to further extrapolate the rat model findings of above to a human subject, the expression of profilin-1 in diabetic aorta tissue samples was studied. The study comprised the Western blot analyses shown in FIG. 2 in which profilin-1 levels were significantly elevated or increased in the aorta endothelium of 5 month diabetic rats with n equal to 5 and a p-value of less than 0.05. For human aorta specimens obtained from diabetic donors, the results also showed elevated profilin-1 levels with n equal to 3 and a p-value less than 0.05. FIG. 2 also shows that profilin-1 levels were elevated in diabetic glomeruli, although the elevation was not shown to be statistically significant. In addition, immunofluorescence analyses in rat aorta tissue sections confirmed that profilin-1 was increased in diabetic specimens with a preferential staining of the endothelial layer and staining to a lesser extent in the media. As a result, profilin-1 is shown to be overexpressed in the aortic endothelium in response to DVD (9).

The present example demonstrates that profilin-1 levels are elevated or increased in diabetic endothelium obtained from both human and rat aortas in comparison to respective nondiabetic tissue samples. Staining for profilin-1 by immunofluorescence also confirmed an increase in diabetic aorta samples and further showed preferential staining of the endothelium. The increase in profilin-1 levels in human and diabetic rats underscores the direct role of profilin-1 in EC dysfunction as associated with DM and DVD.

### EXAMPLE II

The role of profilin-1 in EC dysfunction was assessed in RAEC that overexpressed profilin-1. By way of comparison to empty vector-RAEC, profilin-1 overexpressing RAEC showed signs of EC dysfunction including accelerated apoptosis, phosphorylated VASP and leukocyte adhesion through, for example, ICAM-1. With regard to apoptosis, even a modest increase in profilin-1 levels were show to accelerate apoptosis as evaluated by flow cytometry using Annexin-V staining when compared to empty vector-RAEC. Sensitivity to hydrogen peroxide-induced apoptosis, however, was not affected by an overexpression of profilin-1.

The phosphorylation of VASP is known to occur in response to nitric oxide (NO) and has been used as a tool to monitor NO-dependent signaling in situ (19). In the present example, it was found that VASP phosphorylation was significantly decreased in the aorta of diabetic rats using both immunoblot analyses and immunofluorescence (9). These findings show that an overexpression of profilin-1 in RAEC decreases NO-meditation. VASP phosphorylation also significantly decreases in the aorta of diabetic rats as compared to age matched animals. Such studies were similarly based on immunoblot analyses and immunofluorescence.

Leukocyte adhesion to endothelium tissues through adhesion molecules including ICAM-1 was shown in response to cytokines and atherosclerosis (20 and 21). It was found that ICAM-1 proteins levels were increased in profilin-1 overexpressing RAEC as compared to empty vector-RAEC. Similarly, ICAM-1 levels were increased in the aortic endothelium of diabetic rats. Accordingly, an association exists between profilin-1 and ICAM-1 as both are increased in RAEC overexpressing profilin-1 and in the diabetic aorta.

The present example also shows that abnormalities caused by forced profilin-1 expression in phosphorylated VASP and leukocyte adhesion were recapitulated in the diabetic aorta. This finding indicates that profilin-1 increases in DM play a role in diabetic linked EC dysfunction. As a whole, the studies of accelerated apoptosis, phosphorylated VASP and leukocyte adhesion through ICAM-1 indicate that profilin-1 mediates endothelial dysfunction via multiple mechanisms and that profilin-1 represents a rationale therapeutic target for prevention or management of CVD.

### EXAMPLE III

In the present example, it is determined that exposure to LDL and oxysterol is able to mediate profilin-1 increases in RAEC. This result, however, was not observed with high concentrations of 30 mM of glucose. These findings are of particular interest as it is known that LDL is a major risk factor for atherosclerosis and that oxysterol is abundant within human atherosclerotic plaque (22 and 23). It was further shown that cytokine TNF-α and recombinant profilin-1 were unable to upregulate profilin-1 levels.

Conversely, these studies indicate that only LDL and, in particular, oxidized LDL were able to increase profilin-1 at the MRNA and protein levels. Given that LDL is the major cholesterol-carrying particle in vascular circulation, the direct effects of oxysterol and native cholesterol were studied. These studies demonstrated that only oxysterol could upregulate profilin-1 in RAEC. Accordingly, profilin-1 can be regulated specifically by the well established CVD risk factors of LDL and oxysterol with both of these factors being prevalent in human atherosclerotic plaque (23).

Profilin-1 also operates downstream of LDL and oxysterol in mediating CVD. These study findings are pertinent to numerous applications for disease screening and treatment including screening or treatment of such diseases or conditions as CVD, hypertension, atherosclerosis, hyperlipidemia or DVD. For example, extracellular profilin-1 can be detected on the endothelium of aorta, femoral and carotid arteries in either apoE-null or Ldlr-null mice, which are validated atherosclerosis models.

### EXAMPLE IV

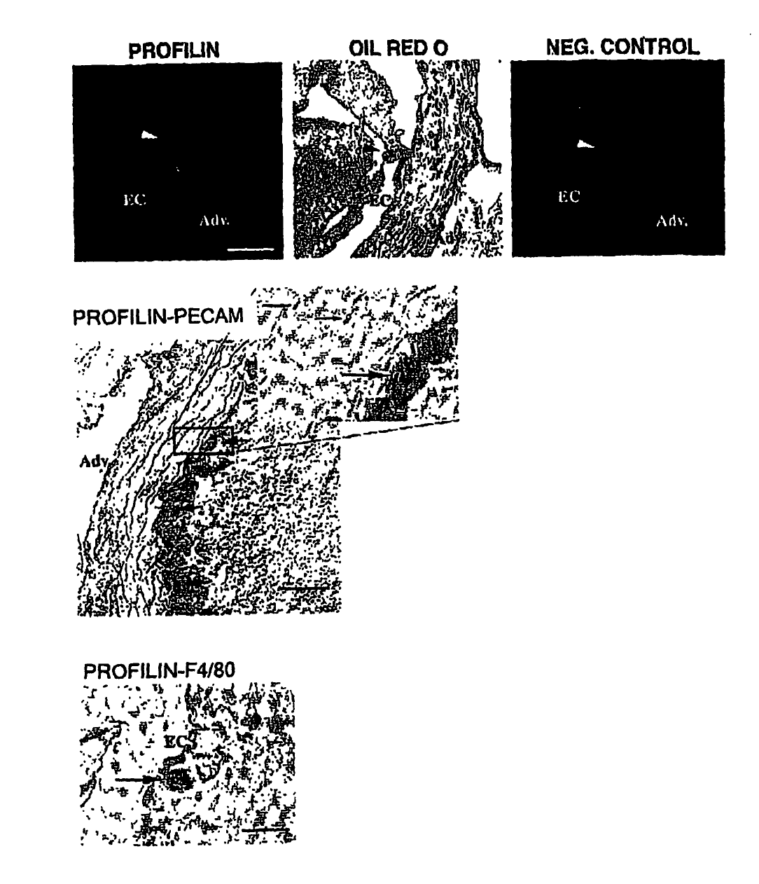

Profilin-1 expression was studied on atherosclerotic plaque in a vessel and compared to disease-free adjacent areas therein. The results of this study are illustrated in FIG. 3. FIG. 3 shows, particularly, stained sections cut from the aortic sinus of apoE-null mice. The sample population per group in this study was 6. The apoE-null mouse is a validated model of atherosclerosis and can also be applied to studies of human atherosclerosis (24). The model displayed elevated levels of VLDL and LDL, which are further enhanced by DM (24 and 25). Thus, profilin-1 expression was shown to be dramatically upregulated in atherosclerotic lesions as compared to adjacent lesion-free areas. Moreover, double-labeling with the endothelial marker PECAM-1 revealed that profilin-1 was highly expressed in EC within the plaque. Double-labeling with monocyte-macrophage marker F4-80 also indicated that profilin-1 was overexpressed in macrophage infiltrating the plaque.

These findings demonstrate the direct role of profilin-1 in atherosclerotic plaque development. Such plaque development can be blunted by administering or delivering an siRNA based therapeutic method such as described above. For instance, a synthetic siRNA can be engineered to downregulate profilin-1 gene transcription. An siRNA based protocol could also be applied to blunt excess profilin-1 expression in vessels that are commonly affected by atherosclerosis such as coronary, carotid and femoral arteries. Delivery of siRNA or synthetic siRNA to a single target artery is achieved by standard angiographic procedures like those currently used to deliver fibrinolytic agents. An exemplary gene therapy method according to the invention is preferred over siRNA based methods when multiple vessels are affected by a disease or condition associated with EC dysfunction or when a systemic delivery approach would be desirable. Such a gene therapy protocol can, as previously described, comprise modifying, substituting, regulating or interfering with a gene associated with profilin-1 activity or function.

### REFERENCES

1. Cosentino F and Luscher T F. Endothelial dysfunction in diabetes mellitus. J. Cardiovasc. Pharmacol. 32: S54-S61, 1998.
2. Garcia S F, Virag L, Jagtap P, Szabo E, Mabley J G, Liaudet L, Marton A, Hoyt D G, Murthy K G, Salzman A L, Southan G J and Szabo C. Diabetic endothelial dysfunction: the role of poly(ADP-ribose) polymerase activation. Nature Med. 7: 108-113, 2001.
3. Beckman J A, Creager M A and Libby P. Diabetes and atherosclerosis: epidemiology, pathophysiology, and management. JAMA. 287: 2570-2581, 2002.
4. Haffner S M. Lipoprotein disorders associated with type 2 diabetes mellitus and insulin resistance. Am. J. Cardiol. 90: 55i-61i, 2002.
5. Haffner S M, Lehto S, Ronnemaa T, Pyorala K and Laakso M. Mortality from coronary heart disease in subjects with type 2 diabetes and in nondiabetic subjects with and without prior myocardial infarction. N. Engl. J. Med. 339: 229-234, 1998.
6. Steinberg H O, Bayazeed B, Hook G, Johnson A, Cronin J and Baron A D. Endothelial dysfunction is associated with cholesterol levels in the high normal range in humans. Circulation. 96: 3287-3293, 1997.
7. Lorenzi M and Gerhardinger C. Early cellular and molecular changes induced by diabetes in the retina. Diabetologia 44: 791-804, 2001.
8. Tilton R G, Chang K, Pugliese G, Eades D V D, Province M A, Sherman W R, Kilo C and Williamson J R. Prevention of hemodynamic and vascular albumin filtration changes in diabetic rats by aldose reductase inhibitors. Diabetes 38: 1258-1270, 1989.
9. Romeo G, Frangioni J V and Kazlauskas A. Profilin acts downstream of LDL to mediate diabetic endothelial cell dysfunction. FASEB. J. 18: 725-727, 2004.
10. Pugliese G, Pricci F, Mene P, Romeo G, Nofroni I, Giannini S, Cresci B, Galli G, Rotella C M, Di Mario U and Pugliese F. High glucose level unmasks a genetic predisposition to enhanced extracellular matrix production in mesangial cells from rats of the Milan normotensive strain. J. Am. Soc. Nephrol. 8: 406-414, 1997.
11. Podesta F, Romeo G, Liu W H, Krajewski S, Reed J C, Gerhardinger C and Lorenzi M. Bax is increased in the retina of diabetic subjects and is associated with pericyte apoptosis in vivo and in vitro. Am. J. Pathol. 156: 1025-1032, 2000.
12. Frangioni J V, LaRiccia L M, Cantley L C and Montminy M R. Minimal activators that bind to the KIX domain of p300/CBP identified by phage display screening. Nature Biotechnol. 18: 1080-1085, 2000.
13. Voss S D, DeGrand A M, Romeo G R, Cantley L C and Frangioni J V. An integrated vector system for cellular studies of phage display-derived peptides. Anal. Biochem. 308: 364-372, 2002.
14. Garcia-Cardena G, Comander J, Anderson K R, Blackman B R and Gimbrone M A Jr. Biomechanical activation of vascular endothelium as a determinant of its functional phenotype. Proc. Natl. Acad. Sci. U.S.A. 98: 4478-4485, 2001.
15. Passerini A G, Polacek D C, Shi C, Francesco N M, Manduchi E, Grant G R, Pritchard W F, Powell S, Chang G Y, Davies P F and Stoeckert C J Jr. Coexisting proinflammatory and antioxidative endothelial transcription profiles in a disturbed flow region of the adult porcine aorta. Proc. Natl. Acad. Sci. U.S.A. 101 (8): 2482-2487, 2004.
16. Carlson L, Nystrom L E, Sundkvist I, Markey F and Lindberg U. Actin polimerizability is influenced by profilin, a low molecular weight protein in non-muscle cells. J. Mol. Biol. 115: 465-483, 1997.
17. Didry D, Carlier M F and Pantaloni D. Sinergy between actin depolymerizing factor-cofilin and profilin in increasing actin filament turnover. J. Biol. Chem. 273: 25602-25611, 1998.
18. Witke W, Podtelejnikov A V, DiNardo A, Sutherland J D, Gurniak C B, Dotti C and Mann M. In mouse brain profilin I and profilin II associate with regulators of the endocytic pathway and actin assembly. EMBO. J. 17: 967-976, 1998.
19. Smolenski A, Bachmann C, Reinhard K, Honig-Liedl P, Jarchau T, Hoschuetzky H and Walter U. Analysis and regulation of vasodilator-stimulated phosphoprotein serine 239 phosphorylation in vitro and in intact cells using a phosphospecific monoclonal antibody. J. Biol. Chem. 273: 20029-20035, 1998.
20. Osborn L, Hession C, Tizard R, Vassallo C, Luhowskyj S, Chi-Rosso G and Lobb R. Direct expression cloning of vascular cell adhesion molecule 1, a cytokine-induced endothelial protein that binds to lymphocytes. Cell. 59: 1203-1211, 1989.
21. Cybulsky M I and Gimbrone M A, Jr. Endothelial expression of a mononuclear leukocyte adhesion molecule during atherogenesis. Sci. 251: 788-791, 1991.
22. Steinberg D and Gotto A M Jr. Preventing coronary artery disease by lowering cholesterol levels: fifty years from bench to bedside. JAMA. 282: 2043-2050, 1999.
23. Brown A J, Leong S L, Dean R T and Jessup W. 7-Hydroperoxycholesterol and its products in oxidized low density lipoprotein and human atherosclerotic plaque. J. Lipid. Res. 38: 1730-1745, 1997.
24. Plump A S, Smith J D, Hayek T, Aalto-Setala K, Walsh A, Verstuyft J G, Rubin E M and Breslow J L. Severe hypercholesterolemia and atherosclerosis in apolipoprotein E-deficient mice created by homologous recombination in ES cells. Cell. 71: 343-353, 1992.
25. Park L, Raman K G, Lee K J, Lu Y, Chow W S, Stern D, Schmidt A M and Ferran L J Jr. Suppression of accelerated diabetic atherosclerosis by the soluble receptor for advanced glycation endproducts. Nature Med. 4: 1025-1031, 1998.

While the present invention has been described herein in conjunction with a preferred embodiment, a person of ordinary skill within the art, after reading the foregoing specification, will be able to effect various changes, substitutions of equivalents and alterations to the methods, protocols or compositions set forth herein. Each of the embodiments described above can further have included or incorporated therewith such variations as disclosed with regard to any or all of the other embodiments. It is therefore intended that protection granted by Letter Patent hereon be limited in breadth only by the definitions contained in the appended claims and any equivalents thereof.

What is claimed is:

1. A method of determining the state of a disease or condition associated with endothelial cell dysfunction in a subject, the method comprising:
providing a subject suffering from or thought to be at risk of suffering from a disease or condition associated with endothelial cell dysfunction;
determining a level of profilin-1 at a site in the subject; and
relating the level to the state of the disease or condition.

2. The method of claim 1, wherein an increased level of profilin-1 indicates a positive diagnosis for the disease or condition.

3. The method of claim 1, wherein the disease or condition is diabetic vascular disease.

4. The method of claim 1, wherein the disease or condition is atherosclerosis.

5. The method of claim 1, wherein the disease or condition is a vascular disease selected from the group consisting of peripheral vascular disease, cerebrovascular disease, cardiovascular disease and any combination thereof.

6. The method of claim 1, wherein the site is associated with a vascular circulation sample from the subject.

7. The method of claim 6, wherein the vascular circulation sample is a serum sample.

8. The method of claim 6, wherein the vascular circulation sample is a plasma sample.

9. The method of claim 1, wherein the site is associated with a tissue sample from the subject.

10. The method of claim 9, wherein the tissue sample from the subject is an aorta tissue sample.

11. The method of claim 9, wherein the tissue sample from the subject is a retina tissue sample.

12. A kit for performing the method of determining the state of a disease or condition associated with endothelial cell dysfunction in claim 1, the kit comprising:
an indicator, wherein the indicator is capable of indicating the level of profilin-1; and
a device, wherein the device is capable of qualitatively or quantitatively monitoring the indicator for indication of the level of profilin-1.

13. A method for prophylaxis or treatment of a disease or condition associated with endothelial cell dysfunction in a subject, the method comprising:
providing a subject suffering from or thought to be at risk of suffering from a disease or condition associated with endothelial cell dysfunction;
administering to the subject a therapeutic composition or protocol to inhibit an activity or function of profilin-1, wherein inhibiting the activity or function of profilin-1 prevents or treats the disease or condition.

14. The method of claim 13, wherein a pharmaceutically acceptable carrier comprises the therapeutic composition.

15. The method of claim 13, wherein the therapeutic protocol comprises modifying, substituting, regulating or interfering with a gene associated with profilin-1 activity or function.

16. The method of claim 13, wherein the therapeutic protocol comprises downregulating gene transcription associated with profilin-1 synthesis.

17. The method of claim 13, wherein the disease or condition is diabetic vascular disease.

18. The method of claim 13, wherein the disease or condition is atherosclerosis.

19. The method of claim 13, wherein the disease or condition is a vascular disease selected from the group consisting of cerebrovascular disease, cardiovascular disease, peripheral vascular disease and any combination thereof.

20. A therapeutic composition for prophylaxis or treatment of a disease or condition associated with endothelial cell dysfunction in a subject, the therapeutic composition comprising:
a inhibitor capable of inhibiting an activity or function of profilin-1; and
a pharmaceutically acceptable carrier comprising the inhibitor.

21. The therapeutic composition of claim 20, wherein the disease or condition is diabetic vascular disease.

22. The therapeutic composition of claim 20, wherein the disease or condition is atherosclerosis.

23. The therapeutic composition of claim 20, wherein the disease or condition is a vascular disease selected from the group consisting of peripheral vascular disease, cerebrovascular disease and cardiovascular disease and any combination thereof.

24. A therapeutic protocol for prophylaxis or treatment of a disease or condition associated with endothelial cell dysfunction in a subject, the therapeutic protocol comprising:
providing a subject suffering from or thought to be at risk of suffering from a disease or condition associated with endothelial cell dysfunction; and
administering a small interfering RNA to the subject, wherein the small interfering RNA downregulates gene transcription associated with profilin-1 to prevent or treat the disease or condition.

25. The therapeutic protocol of claim 24, wherein the small interfering RNA is a synthetic small interfering RNA.

26. The therapeutic protocol of claim 24 or 25, wherein the small interfering RNA is delivered via a lentivirus.

27. The therapeutic protocol of claim 24 or 25, wherein the small interfering RNA is delivered to an artery selected from the group consisting of coronary, carotid, femoral and a combination thereof.
